(19) United States
(12) Patent Application Publication
(10) Pub. No.: US 2011/0295141 A1
(43) Pub. Date: Dec. 1, 2011

(54) METHODS AND SYSTEMS FOR ENDOBRONCHIAL DIAGNOSTICS

(75) Inventors: Srikanth Radhakrishnan, Cupertino, CA (US); Ryan Olivera, Granite Bay, CA (US); Niyazi Beyhan, Santa Clara, CA (US); Surag Mantri, Sunnyvale, CA (US); Lutz Freitag, Hemer (DE)

(73) Assignee: Pulmonx Corporation, Redwood City, CA (US)

(21) Appl. No.: 12/954,499

(22) Filed: Nov. 24, 2010

Related U.S. Application Data
(60) Provisional application No. 61/289,868, filed on Dec. 23, 2009.

(51) Int. Cl. A61B 5/08

(52) U.S. Cl. 600/532

(57) ABSTRACT

A method for assessing lung function in a patient is disclosed. The method comprises isolating a lung compartment. Thereafter, in one embodiment, an inhaled gas of known composition is introduced into the lung and compared to the composition of the exhaled gas. Alternatively, accumulated CO₂ content is measured within the isolated lung compartment over time, and compared to a baseline CO₂ content. Alternatively, a change in pressure of an isolated lung compartment may be monitored. Alternatively, the magnitude of the range of CO₂ values in an isolated lung compartment can be compared to a predetermined threshold. Any of the results obtained via these alternative embodiments may be used to determine lung function.

### CROSS-REFERENCE TO RELATED APPLICATION

This application claims the benefit of Provisional Application No. 61/289,868 (Attorney Docket No. 017534-004700US), filed on Dec. 23, 2009, the full disclosure of which is incorporated herein by reference.

### BACKGROUND OF THE INVENTION

1. Field of the Invention

This invention relates generally to medical methods and systems and more specifically to methods for assessing the functionality of lung compartments and treating diseased compartments of the lung.

2. Description of the Related Art

Lung diseases are a problem affecting several millions of people. Chronic obstructive pulmonary disease (COPD), for example, is a significant medical problem affecting 16 million people or about 6% of the U.S. population. Lung cancer, as another example, is among the most prevalent forms of cancer, and causes more than 150,000 deaths per year. In general, two types of diagnostic tests are performed on a patient to determine the extent and severity of lung disease: 1) imaging tests and 2) functional tests. Imaging tests, such as chest x-rays, computed tomography (CT) scans, magnetic resonance imaging (MRI), perfusion scans, and bronchograms, provide a good indicator of the location, homogeneity and progression of the diseased tissue. However, these tests do not give a direct indication of how the disease is affecting the patient's overall lung function and respiration capabilities. This can be measured with functional testing, such as spirometry, plethysmography, oxygen saturation, and oxygen consumption stress testing, among others. Together, these diagnostic tests are used to determine the course of treatment for the patient.

However, the diagnostic tests for COPD are limited in the amount and type of information that may be generated. For example, diagnostic imaging may provide information to the physician regarding which lung regions “appear” more diseased, but in fact a region that appears more diseased may actually function better than one that appears less diseased. Similarly, functional testing is performed on the lungs as a whole. Thus, the information provided to the physician is generalized to the whole lung and does not provide information about functionality of individual lung compartments, which may be diseased. Thus, physicians may find it difficult to target interventional treatments to the compartments most in need and to avoid unnecessarily treating compartments that are least in need of treatment. Therefore, in general, using conventional imaging or functional testing, the diseased compartments cannot be differentiated, prioritized for treatment, or assessed after treatment for their level of response to therapy.

One particular need is the diagnosis of lung compartments that would be candidates for lung volume reduction (LVR). LVR typically involves resecting diseased portions of the lung. Resection of diseased portions of the lungs both promotes expansion of the non-diseased regions of the lung and decreases the portion of air which is inhaled into the lungs but is not used to transfer oxygen to the blood. Lung reduction is conventionally performed in open chest or thoracoscopic procedures where the lung is resected, typically using stapling devices having integral cutting blades. While effective in many cases, conventional lung reduction surgery is significantly traumatic to the patient, even when thoracoscopic procedures are employed. Further, such procedures often result in the unintentional removal of relatively healthy lung tissue or leaving behind of relatively diseased tissue, and frequently result in air leakage or infection.

One of the emerging methods of lung volume reduction involves the endoscopic introduction of implants into pulmonary passageways. Such a method and implant is described in U.S. patent application Ser. No. 11/682,986. The implants will typically restrict air flow in the inhalation direction, causing the adjoining lung compartment to collapse over time. This method has been suggested as an effective approach for treating lung compartments that are not subject to collateral ventilation.

There is a need for a quick and convenient method of determining whether a diseased lung portion is suitable for placement of an implant for effective LVR. This depends on the presence of collateral channels which often reduce the effectiveness of LVR using an implant. Collateral channels are sometimes naturally present in the lungs because of gaps in the natural membranes separating the lobes and segments. In many cases, however, COPD manifests itself in the formation of a large number of collateral channels caused by rupture of the air sacs because of hyperinflation, or by destruction and weakening of alveolar tissue, leading to many pathways for air to flow between lung segments. The presence of these collateral channels impedes LVR treatment using one-way valves and implants to induce collapse of a lung segment. This is because the collateral channels allow air to flow into the lung compartment from an adjacent compartment. This replenishes the air in the compartment and prevents the lung compartment from collapsing. If collateral channels exist, options other than LVR may be explored. The selection of this method of LVR as a treatment option would thus be based on the presence or absence of collateral channels. There is thus a need to determine the presence of collateral channels, or at least ventilation due to collateral channels (i.e., collateral ventilation).

Further, if collateral channels are present, regardless of whether LVR is chosen as a treatment option, it would be further desirable to discern their ancillary characteristics, such as the extent of a compartment's hyperinflation, the size of the collateral channels, and the perfusion rate through the pathways and the particular lobes or segments of the lung that are connected by these pathways. Discerning such characteristics enables the treatment to be tailored to the nature and quality of the collateral channels. For example, depending on the nature and size of the collateral channels, different agents may have to be used to seal the collateral channels. There is therefore a need for accurately determining the presence of collateral pathways as well as the characteristics of such pathways.

Various methods for determining collateral ventilation have been proposed. For example, Morrel et al. (1994) analyzed gas compositions in lungs of emphysematous patients. After occluding a lung compartment, they introduced an O₂—He mixture as a breathing gas into the isolated lung compartments. The helium gas content in the isolated lung was measured, as was the CO₂ content. They correlated the rise of helium within the isolated compartment to the extent of collateral ventilation. They also measured significantly lower P_{CO2}, in the occluded segments in emphysematous patients, but could not conclude definitively on the state of collateral ventilation using these measurements.

More recently, a number of methods for determining collateral ventilation have been disclosed, as in co-pending U.S. Published Patent Applications 2003/0051733, 2003/0055331, 2007/0142742, 2006/0264772 and 2008/0200797. U.S. Patent Application 2003/0055331 discloses a non-invasive method of diagnosing the presence of disease in various parts of the lung using imaging and computerized integration of the imaging data. The methods described help determine which lung portions are the most severely affected and which lung channels will respond effectively to isolation treatment.

An endobronchial catheter-based diagnostic system is disclosed in U.S. Patent Application 2003/0051733, wherein the catheter uses an occlusion member to isolate a lung segment and the instrumentation is used to gather data such as changes in pressure and volume of inhaled/exhaled air. The data collected is used to diagnose the extent of hyperinflation, lung compliance, etc., in the lung segment. The Application also discloses the use of radiopaque gas and polarized gas that would enable the presence of collateral channels to be identified using radiant imaging and MRI, respectively. A similar method is disclosed in U.S. Patent Application 2008/0027343 in which an isolation catheter is used to isolate a targeted lung compartment and pressure changes therein are sensed to detect the extent of collateral ventilation.

U.S. Patent Application 2007/0142742 discloses further methods of diagnosis of collateral ventilation in a lung using pressure/volume changes in an isolated lung compartment with and without a valve installed therein. It further discloses detecting the propagation of an inert gas such as helium outside the isolated lung compartment to indicate the presence of such collateral channels. These measurements are targeted at quantitative measurements of the extent of collateral flow prevalent in the lung region of interest. Similarly, U.S. Patent Application 2005/0288702 to McGurk et al. discloses a method by which air containing a marker gas is inhaled by the patient and its presence detected in the isolated lung compartment to detect the presence of collateral ventilation.

A method for detecting the extent of hyperinflation in an isolated lung compartment is disclosed in U.S. Patent Application 2006/0264772, wherein the drop in air exhaled through a one-way valve is monitored. The Application also discloses methods of measuring lung compliance and the extent of blood flow and volumetric blood flow to a particular lung segment, the latter method using a tracer gas that would be dissolved in the blood. U.S. Patent Application 2008/0200797 discloses a method of temporarily isolating several feeding channels of a portion of a lung to observe its effects on lung function. The Application also discloses monitoring of CO2 and oxygen within the isolated lung compartment to indicate the efficiency of gas exchange within the compartment.

A slightly different approach to measuring collateral ventilation is disclosed in U.S. Patent Application 2006/0276807. Here, the airway leading to the section of lung to be evaluated is sealed using a catheter with a sealing element and a sudden pressurization or evacuation is applied. Change of pressure within the isolated section is sensed through the catheter. Presence of collateral ventilation is indicated by a change in pressure of the isolated section after the airway is pressurized or evacuated.

Alternative methods and devices for assessing collateral ventilation and other lung function parameters are still being sought. Ideally, such methods and devices may allow a user to choose a diagnostic test that is best tailored to an individual patient's needs. For example, it would be desirable to be able to acquire more quantitative information on the nature and extent of collateral flow between different lung compartments. It would also be desirable to be able to better determine spatial location of collateral pathways within a lung, thereby reducing the treatment cycle time and damage to healthy tissue. At least some of these objectives will be met by the embodiments described herein.

### BRIEF SUMMARY OF THE INVENTION

In one aspect of the present invention, a method for assessing lung function in a patient may first involve introducing a catheter comprising a distal end and a proximal end with at least one lumen therebetween into an airway leading to a targeted compartment of one of the patient's lungs. The distal end of the catheter may include an expandable occluding element configured to sealingly engage a wall of the airway. The proximal end of the catheter may include an inflation port to expand the occluding element and an access port fluidly connected to the lumen. The method may further involve: isolating the targeted lung compartment by expanding the occluding element; introducing into the lung an inhaled gas of known composition; analyzing a composition of an exhaled gas exhaled from the lung; comparing the composition of the exhaled gas to the composition of the inhaled gas; and assessing function of the lung based on the comparison of exhaled and inhaled gases.

In various embodiments, the known composition may include but is not limited to oxygen, methane, carbon monoxide, helium, carbon dioxide and/or sulfur hexafluoride. In one embodiment, the inhaled gas is introduced into the targeted lung compartment. Alternatively, the inhaled gas may be introduced into a lung compartment other than the targeted lung compartment. In one embodiment, the exhaled gas is exhaled from the targeted lung compartment. In an alternative embodiment, the exhaled gas may be exhaled from a lung compartment other than the targeted lung compartment.

In some embodiments, analysis of the gas includes measuring the composition of the exhaled gas. For example, measuring the composition of the exhaled gas may be performed within the targeted lung compartment in some embodiments. Alternatively, the composition of the exhaled gas may be measured outside the targeted lung compartment but within the lung. In yet another embodiment, composition of the exhaled gas may be measured ex-vivo. In one embodiment, the assessing step involves determining a degree of perfusion of the lung. Alternatively or additionally, assessing may involve determining a degree of collateral ventilation in the lung.

In another aspect, a method for assessing lung function in a patient may first involve introducing a catheter as described above into an airway leading to a targeted compartment of one of the patient's lungs. The method may then involve: sampling gases from the lung compartment with the occluding element in an unexpanded configuration to measure a baseline CO2 content of the lung compartment; isolating the lung compartment by expanding the occluding element; measuring accumulated CO2 content within the isolated lung compartment over time; and assessing function of the lung by evaluating a change between the baseline CO2 content and the accumulated CO2 content over time. In some embodiments, the assessing step may include determining a degree of collateral ventilation in the lung.

In another aspect, the invention may include a method for assessing lung function in a patient. This method may involve introducing a catheter with an expandable occluding element into an airway leading to a lung compartment, isolating the lung compartment by expanding the occluding element at the end of an inspiratory cycle, and assessing lung function by monitoring a change in pressure within the isolated lung compartment over a period of time to measure a parameter that indicates lung function. In some embodiments, the parameter may include a rate of perfusion between the isolated lung compartment and a second lung compartment. Additionally or alternatively, the parameter may include a resistance of collateral channels between the isolated lung compartment and a second lung compartment.

In another aspect, a method for assessing lung function in a patient may include: introducing a catheter with an expandable occluding element into an airway leading to a targeted lung compartment; isolating the targeted lung compartment by expanding the occluding element; obtaining a range of CO2 values by measuring CO2 content within the isolated lung compartment over one or more respiratory cycles; and assessing lung function by comparing the magnitude of the range of CO2 values against a predetermined threshold. In some embodiments, the threshold may be established by using population data. Alternatively, the threshold may be obtained from a second lung compartment in the same patient.

In another aspect, a method for assessing lung function in a patient may include: introducing a catheter with an expandable occluding element into an airway leading to a targeted lung compartment; isolating the targeted lung compartment by expanding the occluding element; measuring CO2 content and airflow within the isolated lung compartment over one or more respiratory cycles; and determining a relationship between CO2 content and airflow to determine disease progression.

In another aspect, a device for endobronchial diagnostics may include a catheter and a gas composition measurement device coupled with the catheter to measure composition of at least one gas inhaled into or exhaled out of the lung. The catheter may include a distal end, a proximal end, a sampling lumen and an auxiliary lumen. The distal end may include an expandable occluding element configured to sealingly engage a wall of an airway leading to a targeted compartment of a lung, and the proximal end may include a hub with an inflation port connected to the auxiliary lumen to expand the occluding element and an access port fluidly connected to the sampling lumen wherein the diameter of the sampling lumen is configured to decrease from the proximal end to the distal end.

In some embodiments, the diameter of the sampling lumen may vary continuously between the proximal end and the distal end. Alternatively, the diameter of the sampling lumen may vary discontinuously between the proximal end and the distal end. In some embodiments, the sampling lumen includes a combination of sections varying continuously or discontinuously in diameter. In some embodiments, the gas composition measurement device may be configured to measure at least one gas, including but not limited to oxygen, methane, carbon monoxide, helium, carbon dioxide and/or sulfur hexafluoride.

### BRIEF DESCRIPTION OF THE DRAWINGS

FIG. 1A shows a diagram of an isolation catheter in accordance with an embodiment of the present invention.

FIGS. 1B, 1C and 1D show embodiments of the isolation catheter in which the sampling lumen is configured to have a continuous or discontinuous variation in diameter.

FIG. 2 shows the isolation catheter accessing a lung compartment.

FIG. 3 shows a diagram of a control unit in accordance with an embodiment of the present invention.

FIGS. 4A-4C illustrate the testing of lung compartments in accordance with one embodiment of the invention where differences in CO₂ content are monitored.

FIGS. 5A-5B show another embodiment in which lung function is determined by analyzing the variation of CO₂ content in an isolated compartment over several respiratory cycles.

### DETAILED DESCRIPTION OF THE INVENTION

Although the detailed description contains many specifics, these should not be construed as limiting the scope of the invention but merely as illustrating different examples and aspects of the invention. Various modifications, changes and variations may be made in the arrangement, operation and details of the methods and systems of the present invention disclosed herein without departing from the spirit and scope of the invention as described.

Various methods and systems for targeting, accessing and assessing diseased lung compartments are described herein. Such lung compartments may be an entire lobe of a lung, a segment, a subsegment or even smaller compartments. Assessment is generally achieved by isolating a lung compartment to obtain various measurements to determine lung functionality. Though COPD is mentioned as an example, the applicability of these methods for treatment and diagnosis is not limited to COPD, but can be applicable to any disease of the lung.

The methods are minimally invasive in the sense that the required instruments are introduced orally, and the patient is allowed to breathe normally during the procedures. The methods involve detecting the presence or characteristics (e.g., concentration or pressure) of one or more naturally occurring or introduced gases to determine the presence of collateral ventilation. Naturally occurring gases include those found in the regular breathing cycle (e.g., O₂ and CO₂). Introduced gases include suitable marker gases such as oxygen, helium, methane, carbon monoxide and sulfur hexafluoride, among others. The relative proportion of these gases in the inhaled and exhaled air is used to derive information on the size and extent of collateral channels. One embodiment of the present invention involves introducing air or a tailored mixture of gases into one or more areas of the lung, isolating a targeted lung compartment and then sampling the exhalate from either the targeted lung compartment or the rest of the lung volume to effect measurement. A second embodiment involves restricting inhalatory air into a lung compartment and measuring the concentration of CO₂ buildup in the lung compartment. A third embodiment involves restricting inhalatory air into a lung compartment and measuring the pressure buildup in the compartment. A fourth embodiment involves restricting inhalatory air into a specific lung compartment and determining whether the rate of change of CO₂ approximates a known concentration of CO₂ in alveolar gas.

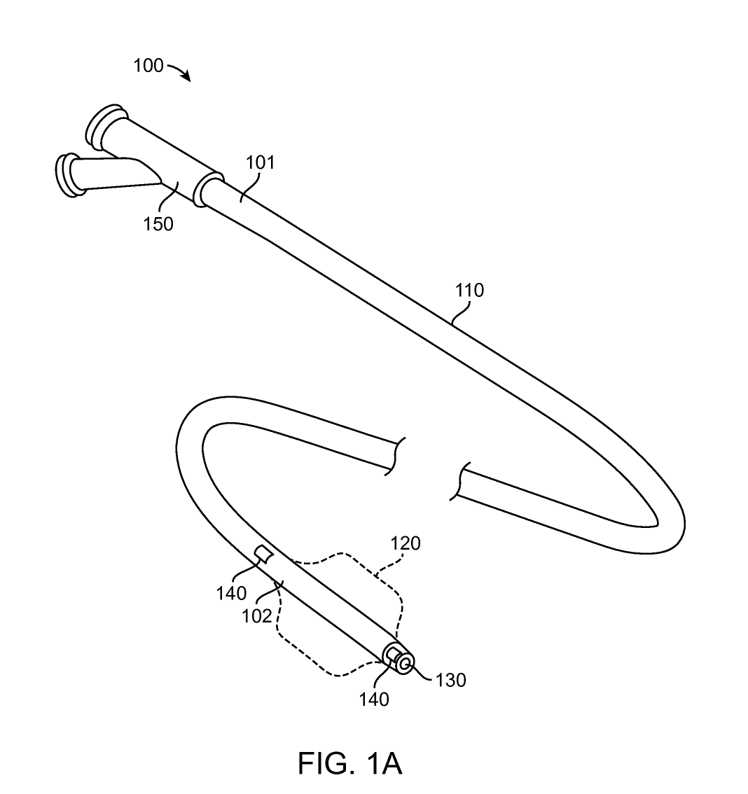

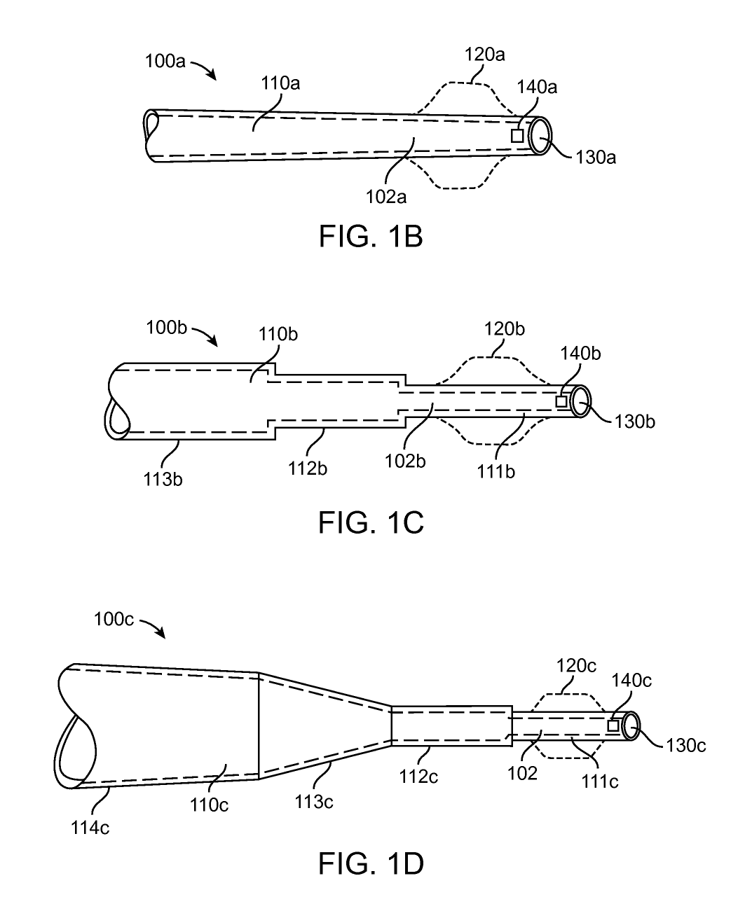

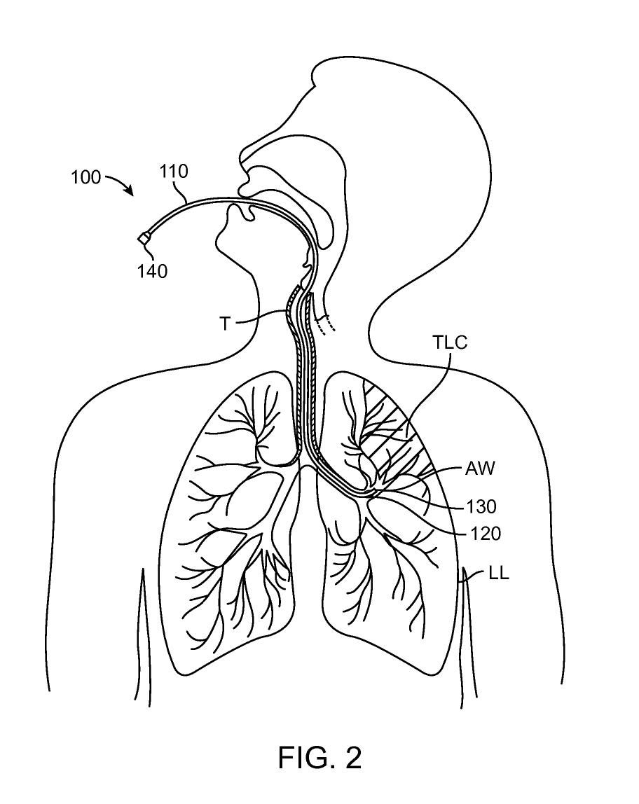

Turning to the figures, in each of the present embodiments, isolation of the lung comprises sealingly engaging a distal end of a catheter in an airway feeding a lung compartment, as shown in FIGS. 1A and 2. Such a catheter has been disclosed in co-pending published U.S. patent application Ser. No. 10/241733, which is incorporated herein by reference. As shown in FIG. 1A, the catheter 100 comprises a catheter body 110, and an expandable occluding member 120 on the catheter body. The catheter body 110 has a distal end 102, a proximal end 101, and at least one lumen 130, or alternatively multiple lumens, extending from a location at or near the distal end to a location at or near the proximal end. The proximal end of catheter 100 is configured to be coupled with an external control unit (not shown), and optionally comprises an inflation port (not shown). The distal end of catheter 100 is adapted to be advanced through a body passageway such as a lung airway. The expandable occluding member 120 is disposed near the distal end of the catheter body and is adapted to be expanded in the airway which feeds the targeted lung compartment. The lumen 130 of the catheter 100 may be of uniform cross-section as shown in FIG. 1A.

In alternative embodiments shown in FIGS. 1B 1C and 1D, the catheter lumen (and, optionally, the corresponding catheter body) is configured to offer minimal resistance to airflow during exhalation and sampling. In the absence of a variable diameter lumen that is shown in FIGS. 1B, 1C and 1D, a typical uniformly small lumen catheter would add resistance to the air flow during exhalation. The variable diameter lumen catheter reduces this catheter resistance, which improves the accuracy of the measurements and makes it easier for the patient to exhale. Thus, in one embodiment shown in FIG. 1B, the catheter body 110a and catheter lumen 130a, have a diameter that gradually tapers from being broader at the proximal end (not shown) to narrower at the distal end 102a. Of course, this embodiment also comprises the balloon 120a and one or more sensors 140a. In another embodiment shown in FIG. 1C, the diameter of the catheter body 110b and lumen 130b may reduce in stages from being broader at the proximal portion to narrower at the distal end 102b. For example, the portion 111b of the catheter body is located at the distal end 130b. Proximal to portion 111b is portion 112b, whose body and lumen are of a larger diameter than portion 111b. Proximal to portion 112b is portion 113b, whose body and lumen are of a larger diameter than portion 112b. The other characteristics of this catheter, including the balloon 120b and the one or more sensors 140b, are similar to those described above.

In another embodiment shown in FIG. 1D, the catheter may have a combination of sections of varying degree of taper as well as of different uniform lumen diameters; thereby offering no additional resistance by the catheter. In the embodiment shown in FIG. 1D, for example, the distal end 102c comprises portion 111c. The catheter body 110c and lumen 130c comprise a uniform diameter in this portion. Portion 111c is configured to be held within a bronchoscope (not shown). Immediately proximal to that distal portion is portion 112c, which is configured to engage with the valve of the bronchoscope. Thereafter, there is a portion 113c, which provides a slow transition as the catheter exits the bronchoscope, to a third diameter of portion 114c.

Additionally and optionally, catheter 100 further comprises at least one gas sensor 140 located within or in-line with the lumen 130 for sensing characteristics of various gases in air communicated to and from the lung compartment. The sensors may comprise any suitable sensors or any combination of suitable sensors, and are configured to communicate with control unit 200, or any intermediary. Exemplary sensors include pressure sensors, temperature sensors, air flow sensors, gas-specific sensors, or other types of sensors. As shown in FIG. 1A, the sensors 140 may be located near the distal end 102 of the catheter 100. Alternatively, the sensors 140 may be located at any one or more points along the catheter 100, or in-line with the catheter and within the control unit with one or more measuring components.

As shown in FIG. 2, at least a distal portion of the catheter body 110 is adapted to be advanced into and through the trachea (T). The catheter body 110 may optionally be introduced through or over an introducing device such as a bronchoscope. The distal end 102 of the catheter 100 can then be directed to a lung lobe (LL) to reach an airway (AW) which feeds a targeted lung compartment (TLC), which is to be assessed. When the occluding member 120 is expanded in the airway, the corresponding compartment is isolated with access to and from the compartment provided through the lumen 130.

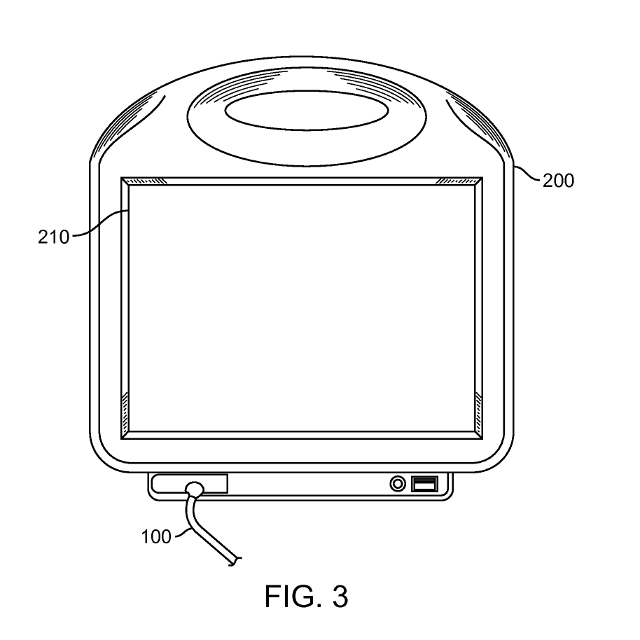

The proximal end of the catheter 100 is configured to be associated with a control unit 200, as shown in FIG. 3. The control unit 200 comprises one or more measuring components (not shown) to measure lung functionality. The measuring components may take many forms and may perform a variety of functions. For example, the components may include a pulmonary mechanics unit, a physiological testing unit, a gas dilution unit, an imaging unit, a mapping unit, a treatment unit, or any other suitable measuring components. The components may be integral with or disposed within the control unit 200. Optionally, control unit 200 may also comprise mechanisms to introduce a gas or a mixture of gases from a gas dilution unit into the isolated lung compartment via one or more catheter lumens. The control unit 200 comprises an interface for receiving input from a user and a display screen 210. The display-screen 210 will optionally be a touch-sensitive screen, and may display preset values. Optionally, the user will input information into the control unit 200 via a touch-sensitive screen mechanism. Additionally and optionally, the control unit may be associated with external display devices such as printers, or chart recorders.

In one embodiment, catheter 100 is introduced into the targeted lung compartment TLC, which is then isolated by inflating the occlusion element 120. Control unit 200 is used to introduce a mixture of gases containing oxygen and one or more marker gases such as methane, carbon monoxide, helium or sulfur hexafluoride into the targeted lung compartment through catheter 100. The patient breathes normally through several respiratory cycles with the TLC exposed to the tailored gas composition.

After the particular gas mixture is introduced into the isolated TLC over several respiratory cycles, analysis of exhaled gas from the rest of the lung (outside the TLC) is carried out using an external sensor that is placed between the occlusion site and the mouth or nose where the expired air is released from the body. The sensor at the mouth or nose could be provided via any suitable apparatus, for example, a mask. The presence of a marker gas, such as helium, detected in the exhaled gas outside the isolated compartment would indicate the presence of collateral channels.

Alternatively, once the TLC is isolated, the gas mixture can be introduced into the rest of the lung from outside the TLC using any suitable method (for example, through the mouth using a mask). Gas from within the TLC would thereafter be analyzed for presence of the markers, to thereby deduce the presence of collateral ventilation.

In another alternative embodiment, the gas mixture may be introduced into the TLC and exhaled gas is sampled from the TLC. If collateral ventilation is present, that would result in a diffusion of some marker gases to locations outside the TLC, thereby resulting in a decrease in concentration of those marker gases in the exhaled volume. Analysis of the change in exhaled gas composition from within the lung compartment over several respiratory cycles would therefore indicate collateral ventilation. Similarly, the tailored gas composition may be introduced to the rest of the lung outside the TLC and exhaled gas from outside the TLC could be analyzed for change in composition over several respiratory cycles.

Additionally or alternatively, besides determining the presence of collateral channels and collateral ventilation, the above embodiment may be used to determine the perfusion efficiency of the collateral channels. Specifically, when gases are introduced into the TLC and are measured from the TLC, the rate of change of the gas composition can be correlated to the perfusion efficiency of the collateral channels feeding the TLC.

Additionally, the method is useful in determining the size of the collateral channels. The gases introduced are intended to vary in molecular size, such that the variation would enable the determination of size and relative proportion of the collateral channels. As molecules diffuse across the collateral channels, their rate of diffusion will depend upon the size of the collateral channel. For example, small molecules will be able to travel across similarly sized collateral channels, whereas larger molecules will be impeded. A determination of the ratio of inhaled to exhaled content of the marker gases would reveal which marker gases were able to travel across, thereby allowing determination of the corresponding size of collateral channels that connect the TLC to the rest of the lung. Additionally and optionally, a feedback control system may be used to vary the ratio of the gaseous components in the mixture. Specifically, the proportion of marker gases in the mixture and the flow rate or pressure at which the gas mixture is introduced may be controlled using the feedback-controlled system, thereby allowing a dynamically adjustable assessment of the sizes and relative proportions of the collateral channels.

In each of the above methods, analysis of gas from within the TLC is performed in-situ using sensors 140 located at the distal end of the catheter. Alternatively, the measurement may be carried out ex-vivo at the control unit 200 by sampling gas within the TLC through catheter lumen 130, or via an external sensor that is placed between the occlusion site and the mouth or nose, where the exhaled air is released out of the body.

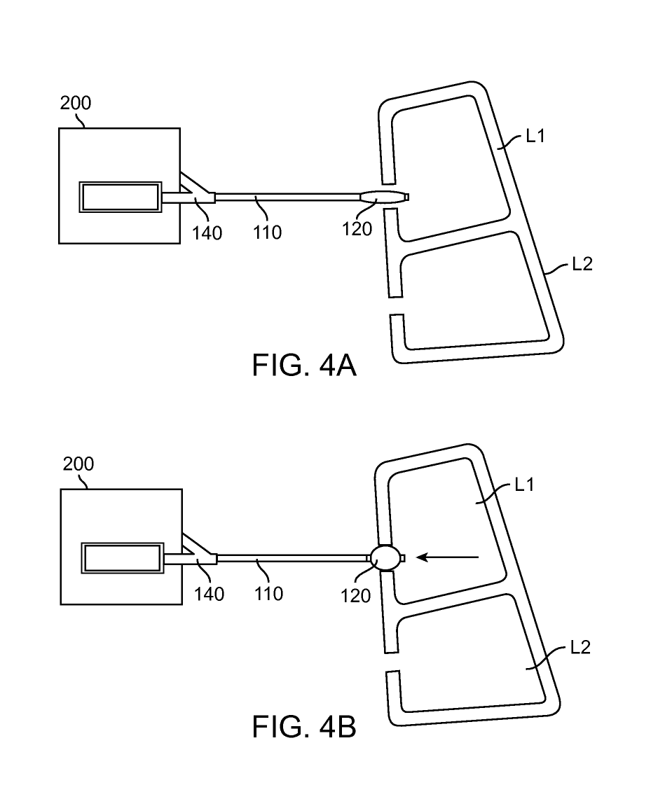

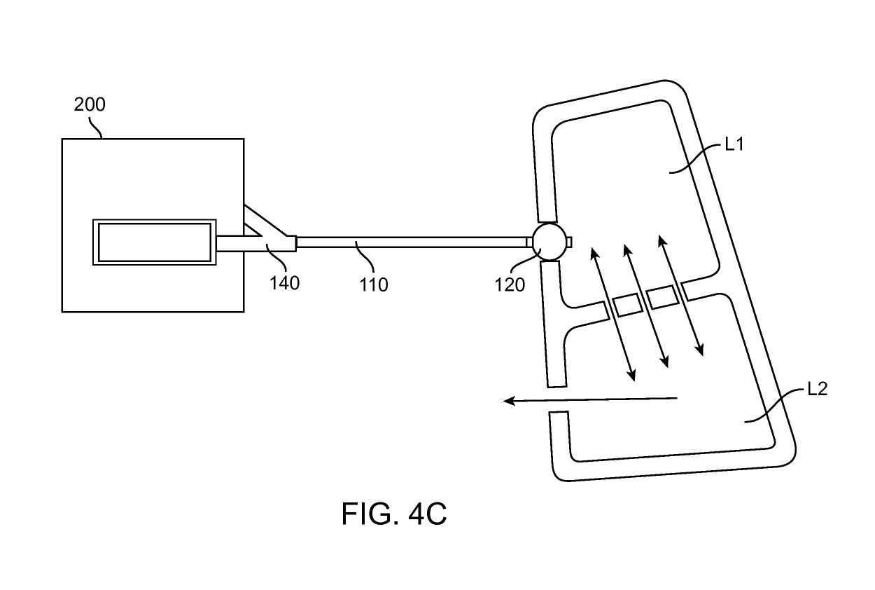

In another embodiment shown in FIGS. 4A to 4C, the presence and nature of collateral channels is determined using a CO₂ sensor to analyze gas within the isolated compartment over time. The patient is allowed to breathe normally and catheter 100 is introduced into the targeted lung compartment L1 as shown in FIG. 4A. With the catheter in position, the CO₂ content in L1 is measured using a sensor located at or near the occluding member 120 over several respiratory cycles to establish a baseline value. Then, the occluding member 120 is expanded to seal the airway, as illustrated in FIGS. 4B and 4C, and external airflow to L1 is ceased. Gas accumulated within the isolated L1 is then analyzed for CO₂ content over a number of respiratory cycles. If collateral channels are not present (FIG. 4B), the CO₂ content within the compartment L1 steadily increases due to effusion from the capillaries in the alveolar tissue. An increasing CO₂ content over time with reference to the baseline value therefore indicates the absence of collateral channels. In contrast, if collateral channels are present, as shown in FIG. 4C, analysis of gas in L1 shows inhibited or no increase in CO₂ content with time over the baseline value, since the CO₂ diffuses out of L1 through the collateral channels. Thus, the rate of increase in CO₂ content can be inversely and numerically correlated to the degree of collateral ventilation.

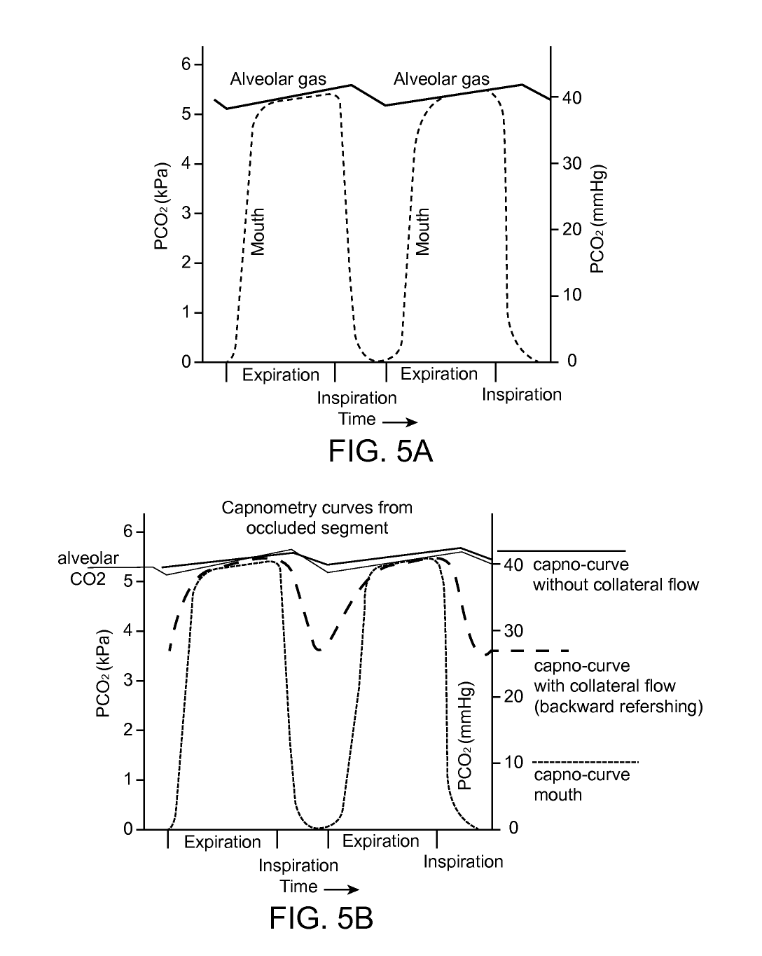

In another embodiment, the catheter 100 with an expandable occluding element 120 is introduced into a body passageway leading to a targeted lung compartment TLC (such as shown in FIG. 2). The targeted lung compartment is then isolated by expanding the occluding element 120 at the end of one inspiratory cycle. Further inspiration into the TLC is then ceased (for example, by blocking passage of inhalation air through lumen 130 of catheter 100) so that the targeted lung compartment is sealed. The pressure within the targeted lung compartment is then monitored over a number of breathing cycles using sensor 140. In normal breathing, pressure in the targeted lung compartment would cycle between positive and negative values. In the absence of collateral ventilation, while air trapped within the isolated targeted lung compartment would diffuse out through tissue, CO₂ would continue to perfuse from the blood in the capillaries over each respiratory cycle, resulting in an overall increase in pressure within the TLC. Thus a steady increase in pressure within the TLC would indicate the relative absence of collateral ventilation. When collateral channels are present, then the rate of pressure increase would be lower than if they are absent, and the rate of the pressure change would be inversely related to the rate of perfusion of the collateral channels. Resistance to perfusion between the TLC and a second adjacent lung compartment can also be measured using this method. For example, a steady increase in pressure would indicate high resistance to perfusion between TLC and a second adjacent lung compartment. In another embodiment, a catheter 100 with expandable occluding element 120 is introduced into a body passageway providing access to a TLC, the body passageway is sealed by expanding occluding element 120, and airflow to the TLC is ceased. Sensor 140 is used to measure alveolar CO₂ content, and one or more additional external sensors at the mouth are used to measure the CO₂ content at the mouth, over several respiratory cycles. Exemplary sensor data gathered using such an embodiment is shown in FIGS. 5A and 5B.

FIG. 5A shows normal lung function, with the variation of alveolar CO₂ content represented by the thick solid line while the expected variation of CO₂ at the mouth is represented by the thin dotted line. The alveolar values (thick solid line) represent the variation in CO₂ content in blood due to gas exchange during respiration, while the corresponding variation at the mouth (thin dotted line) represents the virtual absence of CO₂ in inhaled air versus its attainment of near alveolar values close to the end of a respiration cycle.

The variation of CO₂ content, with and without collateral flow, is illustrated in FIG. 5B. If there is no substantial collateral flow, the CO₂ content after occlusion in the TLC will be similar to the normal alveolar values. This is represented by the thin solid line in FIG. 5B. In contrast, if there is substantial collateral flow, CO₂ content decreases beyond a threshold value due to back flow of air through the collateral channels. This is represented by the thick dashed line in FIG. 5B. The degree of collateral ventilation is determined by examining the extent of variation in CO₂ content beyond the threshold value. The threshold value for determining collateral ventilation can be determined by measurements in a second lung compartment of the same patient without collateral ventilation caused by a diseased condition. Alternatively, the threshold can be determined by measurements in lung compartments of normal healthy subjects in the general population.

In another embodiment, the measurements of CO₂ concentration and flow volume can be used to assess the functional state or destruction of tissue in diseased lung compartments. This is accomplished using the ratio of peak CO₂ concentration to that of the flow volume for each respiratory cycle in a particular lobe. In a normal lung, the peak CO₂ concentration (which typically occurs at the end of the inspiration phase) is high due to good gaseous exchange in the alveolar tissue. This would also be accompanied by a relatively high flow volume compared to a diseased lung portion. Thus, a high CO₂ concentration and a high flow rate signify a normally functioning lung compartment.

In a diseased lung compartment with poor perfusion and/or hyperinflation, the CO₂ levels are also likely to be high (in the same range as found in normal lung); however, the average CO₂ levels are also likely to be high (compared to the average CO₂ levels found in normal lung) due to poor gas exchange or circulation. For these same reasons of poor circulation and exchange, however, the flow volume is likely to be low. Thus, average flow volume in a breathing cycle is a marker of disease progression. By correlating the average flow volume with peak lobar CO₂ levels, lung function can be determined, which can thus lead to identification of diseased and poorly functioning lung compartments and can be used with peak lobar CO₂ levels to determine lung function.

While the above is a complete description of various alternative embodiments, further alternatives, modifications, and equivalents may be used. Therefore, the above description should not be taken as limiting the scope of the invention which is defined by the appended claims.

What is claimed is:

1. A method for assessing lung function in a patient, the method comprising:
introducing a catheter having a distal end, a proximal end and at least one lumen into an airway leading to a targeted compartment of one of the patient's lungs, wherein the distal end comprises an expandable occluding element configured to sealingly engage a wall of the airway, and wherein the proximal end comprises an inflation port to expand the occluding element and an access port fluidly connected to the lumen;
isolating the targeted lung compartment by expanding the occluding element;
introducing into the lung an inhaled gas of known composition;
analyzing a composition of an exhaled gas exhaled from the lung;
comparing the composition of the exhaled gas to the composition of the inhaled gas; and
assessing function of the lung based on the comparison of exhaled and inhaled gases.

2. The method of claim 1, wherein the known composition comprises at least one gas selected from the group consisting of oxygen, methane, carbon monoxide, helium, carbon dioxide and sulfur hexafluoride.

3. The method of claim 1, wherein the inhaled gas is introduced into the targeted lung compartment.

4. The method of claim 1, wherein the inhaled gas is introduced into a lung compartment other than the targeted lung compartment.

5. The method of claim 1, wherein the exhaled gas is exhaled from the targeted lung compartment.

6. The method of claim 1, wherein the exhaled gas is exhaled from a lung compartment other than the targeted lung compartment.

7. The method of claim 1, wherein analyzing comprises measuring the composition of the exhaled gas.

8. The method of claim 7, wherein measuring the composition of the exhaled gas is performed within the targeted lung compartment.

9. The method of claim 7, wherein measuring the composition of the exhaled gas is performed outside the targeted lung compartment.

10. The method of claim 1, wherein analyzing the composition of the exhaled gas is performed within the lung.

11. The method of claim 1, wherein analyzing the composition of the exhaled gas is performed ex-vivo.

12. The method of claim 1, wherein assessing comprises determining a degree of perfusion of the lung.

13. The method of claim 1, wherein assessing comprises determining a degree of collateral ventilation in the lung.

14. A method for assessing lung function in a patient, the method comprising:
introducing a catheter comprising a distal end and a proximal end with at least one lumen therebetween into an airway leading to a targeted compartment of one of the patient's lungs, wherein the distal end comprises an expandable occluding element configured to sealingly engage a wall of the airway, and wherein the proximal end comprises an inflation port to expand the occluding element and an access port fluidly connected to the lumen;
sampling gases from the lung compartment with the occluding element in an unexpanded configuration to measure a baseline CO₂ content of the lung compartment;
isolating the lung compartment by expanding the occluding element;
measuring accumulated CO₂ content within the isolated lung compartment over time; and
assessing function of the lung by evaluating a change between the baseline CO₂ content and the accumulated CO₂ content over time.

15. The method of claim 14, wherein assessing comprises determining a degree of collateral ventilation in the lung.

16. A method for assessing lung function in a patient, the method comprising:
introducing a catheter with an expandable occluding element into an airway leading to a lung compartment;
isolating the lung compartment by expanding the occluding element at the end of an inspiratory cycle; and
assessing lung function by monitoring a change in pressure within the isolated lung compartment over a period of time to measure a parameter that indicates lung function.

17. The method of claim 16, wherein the parameter comprises a rate of perfusion between the isolated lung compartment and a second lung compartment.

18. The method of claim 17, wherein the parameter comprises resistance of collateral channels between the isolated lung compartment and a second lung compartment.

19. A method for assessing lung function in a patient, the method comprising:
introducing a catheter with an expandable occluding element into an airway leading to a targeted lung compartment;
isolating the targeted lung compartment by expanding the occluding element;
obtaining a range of CO₂ values by measuring CO₂ content within the isolated lung compartment over one or more respiratory cycles; and
assessing lung function by comparing the magnitude of the range of CO₂ values against a predetermined threshold.

20. The method of claim 19, wherein assessing comprises determining a degree of collateral ventilation.

21. The method of claim 19, wherein the threshold is established by using population data.

22. The method of claim 19, wherein the threshold is obtained from a second lung compartment in the same patient.

23. A method for assessing lung function in a patient, the method comprising:
introducing a catheter with an expandable occluding element into an airway leading to a targeted lung compartment;
isolating the targeted lung compartment by expanding the occluding element;
measuring CO₂ content and airflow within the isolated lung compartment over one or more respiratory cycles; and
determining a relationship between CO₂ content and airflow to determine disease progression.
